(19) United States
(12) Patent Application Publication
(10) Pub. No.: US 2011/0166328 A1
(43) Pub. Date: Jul. 7, 2011

(54) Avian Antibodies Specific to Influenza Virus and Technologically Simple Methods of Their Manufacture and Use

(76) Inventor: Huan Nguyen, Vestavia Hills, AL (US)

(21) Appl. No.: 13/061,674

(22) PCT Filed: Sep. 2, 2009
(86) PCT No.: PCT/US2009/055767

§ 371 Date: Mar. 1, 2011

Related U.S. Application Data
(60) Provisional application No. 61/093,484, filed on Sep. 2, 2008.

(51) Int. Cl. C07K 16/08

(52) U.S. Cl. 530/387.1

(57) ABSTRACT

Avian antibodies that bind to influenza virus are provided. The disclosed antibodies are inexpensive to produce and are more effective than previously used antibodies against influenza. The present disclosure provides methods of treatment, prevention and diagnosis using such avian antibodies as well as methods of producing the disclosed avian antibodies. Methods of using the disclosed avian antibodies to prevent viral adhesion of influenza to cells are also provided. A novel source of such antibodies is bird eggs from countries in which vaccination against influenza of various bird populations is legally required. The present disclosure solves the problem of the limited supply of antibodies for the diagnosis, treatment, and prevention of influenza.

### FIELD OF THE DISCLOSURE

The present disclosure relates generally to the field of immunology, and more particularly to the use and manufacture of antibodies, such as IgY antibodies, specific to influenza. Influenza antibodies can be used in the prevention of viral adhesion, in the treatment of influenza, the prevention of influenza, in the diagnosis of influenza, and in the detection of influenza virus.

### BACKGROUND

Influenza is a common respiratory disease. The disease is caused by a family of viruses, broadly classified as influenza Types A, B, and C. Type A influenza is the most common and most serious form of the disease in humans. Although generally only fatal in a small fraction of cases, the highly contagious nature of influenza causes a serious burden on human populations in terms of mortality and morbidity. For example, in a typical year from 5-20% of the population of the United States contracts influenza, resulting in over 200,000 hospitalizations and 36,000 deaths. Although most strains of influenza cause mild symptoms, periodically a highly pathogenic strain evolves with a much higher rate of mortality. Two such subtypes currently affecting the human population are the avian influenza strain H5N1 (specifically the highly pathogenic avian influenza virus, HPAIV-H5N1) and the swine influenza strain H1N1 (particularly the novel pandemic influenza A (H1N1) 2009 virus—H1N1/09—also known as “novel H1N1 virus”).

Although antiviral drugs can alleviate the symptoms in some cases of influenza, these are not completely effective and some victims die despite the administration of antiviral drugs. Furthermore, some influenza strains are resistant to antiviral drugs. Although influenza vaccines are manufactured every year, the virus mutates quickly, and manufacturers must guess which subtypes will be prevalent during influenza season in order to start production months ahead of time. Consequently, during some years the vaccine fails to predict one or more circulating influenza strains, and is ineffective. Clearly, there is a long-felt but unmet need for truly effective agents to treat and prevent influenza that can be manufactured quickly and easily.

Avian (bird) flu is caused by influenza A viruses that occur naturally among birds. As mentioned above, one highly pathogenic subtype is HPAIV-H5N1.

Avian flu is a concern not only for humans, but also for wildlife and domesticated birds. Wild birds worldwide carry avian influenza viruses in their intestines, often asymptomatically. Avian influenza is very contagious and can spread rapidly through avian populations. Infected birds shed influenza virus in their saliva, nasal secretions, and feces. Domesticated birds may become infected with avian influenza virus through direct or indirect contact with infected wild birds or other infected poultry. Avian influenza infection in domestic poultry causes two main forms of disease that are distinguished by low and high extremes of virulence. The low pathogenic form may go undetected and usually causes only mild symptoms (such as ruffled feathers and a drop in egg production). However, the highly pathogenic form (such as HPAIV-H5N1) spreads more rapidly through flocks of poultry. This form may cause disease that affects multiple internal organs and has a mortality rate that can reach 90-100%, often within 48 hours.

The H5N1 virus is one version of the influenza A virus commonly found in birds. The H5N1 virus has been documented to be transmitted from avian to human populations. Unlike seasonal influenza, where symptoms of infection are generally not life threatening to most subjects, the disease caused by H5N1 (HPAIV-H5N1 particularly) is far more severe and happens quickly, with pneumonia and multi-organ failure commonly observed. Almost 300 people worldwide have been infected with the H5N1 virus since 2003 and more than half of them have died. Most cases have occurred in previously healthy children and young adults. However, it is possible that the only cases currently being reported are those in the most severely ill people, and that the full range of illness caused by the H5N1 virus has not yet been defined. To date, H5N1 influenza has remained primarily an animal disease but should the virus acquire the ability for sustained transmission among humans, populations will have little immunity to this virus and the potential for an influenza pandemic would have grave consequences for global public health.

Previously, there was no effective treatment for H5N1. Supportive care is important, as it is for annual influenza. It is hypothesized that two antiviral medicines approved for human influenza viruses, oseltamivir and zanamivir, may work in treating H5N1 infection in humans. However, this has not been confirmed, and clinical trials involving subjects with H5N1 infection are needed to confirm the effectiveness of these medications. In addition, influenza viruses can sometimes become resistant to these drugs, so these medications may not always be effective. Furthermore, some adverse reactions to oseltamivir have been reported in children.

The risk from avian influenza is generally low to most people, because the viruses do not usually infect humans. H5N1 is one of the few avian influenza viruses to have crossed the species barrier to infect humans, and it is the most deadly of those that have crossed the barrier. Most cases of H5N1 influenza infection in humans have resulted from direct or indirect contact with infected poultry. So far, the spread of H5N1 virus from person to person has not occurred. Nonetheless, because all influenza viruses have the ability to change, there is concern that H5N1 virus one day could be able to infect humans and spread easily from one person to another. If H5N1 virus were to gain the capacity to spread easily from person to person, a pandemic (worldwide outbreak of disease) could begin. No one can predict when a pandemic might occur. However, experts from around the world are watching the H5N1 situation closely and are preparing for the possibility that the H5N1 virus may acquire the ability to be transmitted from animal (i.e. avian) vectors to humans and from person to person once introduced into the human population.

Porcine influenza (“swine flu”) refers to any strain of influenza that is endemic in pigs. However, the term is most often applied to Type A influenzas that infect both pig and human. Swine flu has been responsible for numerous serious outbreaks during the 20th Century, including the 1918 influenza pandemic, the 1968 influenza pandemic, and the 1976 swine flu outbreak. The first influenza pandemic of the 21st Century (the swine flu pandemic of 2009) was due to a highly contagious strain of H1N1 (H1N1/09) swine influenza. Although not as lethal as H5N1, H1N1/09 is much more contagious, having caused 279,085 laboratory confirmed illnesses and 2818 deaths between April and August of 2009. Like earlier forms of pandemic influenza, H1N1/09 infects healthy adults at a much higher rate than does annual influenza.

The H1N1/09 pandemic is illustrative of the need for rapidly produced agents to combat influenza. Although the new strain was discovered in May of 2009, it is expected that only 45 million doses of the newly formulated vaccine will be available in the U.S. by mid-October, when vaccination is most effective. Although it was initially expected that 120 million doses would be ready by mid-October, the inherent difficulties in developing conventional vaccines against novel strains of influenza have delayed development and manufacture. Although the antiviral drugs oseltamivir and zanamivir can have a beneficial effect, many patients have died despite treatment with antiviral drugs.

Vaccination is one of the most effective ways to combat influenza. However, the common “flu shot” is not believed to be effective against H5N1 or H1N1. Research efforts have led to the development of a vaccine for one of the two known strains of the H5N1 influenza virus in humans and for H1N1/09. The U.S. Government has set a goal to expand domestic influenza vaccine production capacity to be able to produce pandemic influenza vaccines for the entire population within six months of a pandemic declaration. However, at the beginning of a pandemic, the scarcity of pre-pandemic and pandemic influenza vaccine will require that the limited supply be allocated or prioritized for distribution and administration. Furthermore, a pandemic may cause significant mortality and morbidity within six months of detection (for example, it is estimated that over 1800 deaths occurred from H1N1/09 during the first three months of the 2009 Influenza Pandemic and over 180,000 illnesses). In the case of the 2009 influenza pandemic, this goal will not be met.

Because there will likely be a limited supply of vaccine, there is an urgent need to develop new and more economical vaccines. As HPAIV-H5N1 infection has about a 50% mortality rate among humans, there is an urgent need for methods of treating and preventing influenza caused by H5N1 and for improved methods of producing vaccines quickly and economically. As H1N1/09 is already spreading globally and is confirmed to have sickened hundreds of thousands of people (with possibly millions of unconfirmed cases) there is a great need for methods of treating and preventing influenza caused by H1N1 and for improved methods of producing vaccines quickly and economically

Antibodies against influenza have potential applications in the prevention of influenza, the treatment and/or prevention of influenza, the diagnosis of influenza infection, the prevention of viral adhesion of influenza virus to cells, and the detection of influenza virus. Traditionally mammalian polyclonal and monoclonal antibodies have been the source of antibodies for such applications. However, as the use of mammalian antibodies suffer a number of disadvantages making their use in treatment, prevention and diagnostic applications problematic, there are compelling reasons to develop egg yolk antibodies over mammalian antibodies. Development of human monoclonal antibodies (mAbs) against H5N1 influenza haemagglutinin (HA) using Epstein-Barr virus (EBV) immortalization of B cells isolated from patients infected with H5N1 (C. P. Simmons et al., PLoS Med. 4, e178 (2007)), phage display (A. P. Lim et al., Virol. J. 5, 130 (2008)), humanized mAbs (B. J. Hanson et al., Respir. Res. 7, 126 (2006)), and human recombinant Abs (L. Sun et al., PLoS One 4, e5476 (2009)) has been attempted. However, passive immunization based on mAbs, may require a cocktail of mAbs with broader specificity to provide full protection, since mAbs are generally specific for single epitopes.

The present disclosure provides a solution to the problem of producing cost effective treatments and vaccines against influenza viruses, such as H5N1 and H1N1. The present disclosure provides for the use of antibodies from birds (avian antibodies) in the treatment, prevention and diagnosis of influenza. The present disclosure also provides for quick and economical production of such avian antibodies. The avian antibodies produced may be used for the prevention of viral adhesion, the treatment of influenza, the prevention of influenza, the diagnosis of influenza, and the detection of influenza virus. Antibodies of the IgY isotype from birds are particularly useful in these applications.

### SUMMARY

It has been discovered that avian IgY antibodies are highly effective in targeting influenza viruses, and are very effective in protecting subjects from mortality and morbidity caused by influenza. Avian IgY antibodies can be produced quickly and at low expense by merely vaccinating a bird against an antigen (such as an influenza antigen) and harvesting IgY antibodies from the bird's eggs. Eggs from such birds can be obtained commercially in countries in which domesticated egg producing hens are routinely or compulsorily vaccinated against avian diseases, including influenza. In such countries, eggs purchased from supermarkets, wholesalers, or other outlets provide a cheap, immediate, and copious source of influenza-recognizing IgY antibodies.

Although egg IgY has been used to prevent bacterial and viral infections (see the review of A. Larsson and D. Carlander, Ups. J. Med. Sci. 108, 129 (2003)) of the gastrointestinal tract and recently for protection against Pseudomonas aeruginosa infection of the respiratory tract of patients with cystic fibrosis (E. Nilsson et al., Pediatr. Pulmonol. 43, 892 (2008)), so far there have been no attempts to determine the efficacy of using egg IgY against respiratory viruses such as influenza.

The disclosure provides preparations of IgY antibodies that recognize influenza comprising a constituent of a bird egg, wherein the constituent comprises a significant amount of the IgY antibodies. The egg may be from a fowl that has been vaccinated against influenza, for example a laying hen cultivated for egg production. The egg may also have been acquired in an open marketplace, for example in a country that requires all domesticated chickens to be vaccinated against influenza. Methods of making such antibody preparations are also provided, comprising obtaining an egg of a fowl previously immunized against a strain of influenza, and separating an antibody fraction from the yolk. The antibody preparations can be used in a pharmaceutical composition for the prevention or treatment of influenza comprising a therapeutically effective amount of the antibody composition. Methods of treatment and prevention of influenza in a subject are provided, comprising administering to the subject the pharmaceutical composition.

The disclosure provides a viral adhesion inhibitor comprising an adhesion inhibiting effective amount of the antibody preparation. The viral adhesion inhibitor can be used in numerous applications, such as inhibiting the adhesion of an influenza virus to a cell. Methods of inhibiting the adhesion of an influenza virus to a cell can be performed in vivo or ex vivo, depending on the application. In one embodiment, the viral adhesion inhibitor is used as a disinfectant or as a component of a disinfectant.

Reporter antibodies are provided, comprising an IgY conjugated to a detectable reagent, wherein the IgY recognizes a strain of influenza. Such reporter antibodies are useful for example in the detection of influenza viruses and the diagnosis of influenza in a subject. Methods of detecting an influenza virus are provided comprising performing an immunoassay on a sample, the immunoassay comprising contacting a sample with the provided antibody composition.

### BRIEF DESCRIPTION OF THE DRAWINGS

FIG. 1. Protection against infection with A/PR8/34 (H1N1). BALB/c mice were treated with A/PR8/34-specific IgY (anti-PR8 IgY) at 8 hours pre- and 16, 40, and 64 hours post-infection with A/PR8/34 (A); at 8, 32, 56, and 80 hours post-infection (B). Five LD₅₀ of mouse-adapted A/PR8/34 and 50 d of IgY were used for intranasal infection and treatment, respectively. Morbidity (body weight loss) and mortality were monitored daily until recovered animals regained their initial weight. The values are the mean of 5-10 mice in each group. Mortality is expressed as % of mice that survived the lethal infection.

FIG. 2. Protection against infection with A/PR8/34 (H1N1) from a single pretreatment. BALB/c mice were treated with A/PR8/34-specific IgY (anti-PR8 IgY) once at 6 hours before infection (A). Five LD₅₀ of mouse-adapted A/PR8/34 and 50 μl of IgY were used for intranasal infection and treatment, respectively. Morbidity (body weight loss) and mortality were monitored daily until recovered animals regained their initial weight. The values are the mean of 5-10 mice in each group. Mortality is expressed as % of mice that survived the lethal infection. Virus titers in the lungs (TCID₅₀) determined at day 3 after infection in mice treated with A/PR8/34 specific IgY at 6 hours before (−6 hrs) or after (+6 hrs) infection (B). The values are the mean of 8 mice in each group shown in this figure and in FIG. 1, derived from 2 independent experiments.

FIG. 3: Protection against infection with A/Aquatic bird/Korea/W81/2005 (H5N2). BALB/c mice were treated with H5N1-specific IgY [anti-H5N1 IgY or different batch of anti-H5N1 IgY (vn045)] at 6 hours before and 18, 42, and 66 hours after infection with H5N2 virus (Pre- and post-infection treatment, A); at 6, 30, 54, and 78 hours after infection (Post-infection treatment, B); or once at 6 hours before infection (Single pre-infection treatment, C). Five LD₅₀ of mouse-adapted A/Aquatic bird/Korea/W81/2005 (H5N2) virus and 50 μl of IgY were used for intranasal infection and treatment, respectively. Morbidity (body weight loss) and mortality were monitored daily until recovered animals regained their initial weight. The values are the mean of 5-10 mice in each group. Mortality is expressed as % of mice that survived the lethal infection.

FIG. 4: Protection by single treatment against infection with HPAIV H5N1. Morbidity and mortality of BALB/c mice that were treated with H5N1-specific IgY [anti-H5N1 IgY] at 6 hours pre- (−6 h) or post- (+6 h) infection with VN/1203 (H5N1) virus (A—6 mice per group). Ten LD₅₀ of VN/1203 H5N1 virus and 50 μl of H5N1-specific IgY were used for intranasal infection and treatment, respectively. Morbidity (body weight loss) and mortality were monitored daily until recovered animals regained their initial weight. Mortality is expressed as % of mice that survived the lethal infection. Virus titers (EID₅₀) in the lungs were determined on day 3 after infection (B). The values are the mean of 4 mice in each group.

FIG. 5: Protection by multiple treatments against infection with HPAIV H5N1. Morbidity and mortality of BALB/c mice that were treated with H5N1-specific IgY [anti-H5N1 IgY] at 6, 24, 48, and 72 hours after infection (A—5 mice per group). Ten LD₅₀ of VN/1203 H5N1 virus and 50 pd of H5N1-specific IgY were used for intranasal infection and treatment, respectively. Morbidity (body weight loss) and mortality were monitored daily until recovered animals regained their initial weight. Mortality is expressed as % of mice that survived the lethal infection. Virus titers (EID₅₀) in the lungs were determined on day 3 after infection (B). The values are the mean of 4 mice in each group. The group of mice receiving single pre-infection treatment was included as control.

FIG. 6: Induction of anti-IgY Abs and IgY treatment in mice with pre-existing anti-IgY. Endpoint titers (log₂) of anti-IgY in the sera of mice immunized with normal IgY (IgY immunized), treated once intranasally with PR8-specific IgY 8 hours before (anti-PR8 IgY −8 h) or three times after infection (anti-PR8 IgY +8, 32, 56 h) (A); Morbidity and mortality of IgY-immunized mice treated with A/PR8/34-specific IgY (anti-PR8 IgY) before (−6 hr) or after (+6 hr) infection with mouse-adapted A/PR8/34 (B). Morbidity (body weight loss) and mortality were monitored daily until recovered animals regained their initial weight. Mortality is expressed as % of mice that survived the lethal infection. The values are the mean of 5-10 mice in each group.

FIG. 7: Anti-IgY Abs do not block neutralizing activity of virus specific IgY. A/PR8 virus neutralizing activity of A/PR8 specific IgY (anti-PR8 IgY) in the absence of anti-IgY serum was determined by microneutralization assay. VN titer of anti-PR8 IgY is 1:320 at which the viral nuclear protein (NP) was not detected (A). In the presence of anti-IgY serum VN by anti-PR8 IgY was not abrogated by incubation with anti-IgY serum (bottom) or normal serum (upper) (B). VN titer (1:320) of anti-PR8 IgY was used in the assay.

### DETAILED DESCRIPTION

### I. Definitions

The terms “prevention”, “prevent”, “preventing”, “suppression”, “suppress” and “suppressing” as used herein refer to a course of action (such as administering a compound or pharmaceutical composition of the present disclosure) initiated prior to the onset of a clinical manifestation of a disease state or condition so as to prevent or reduce such clinical manifestation of the disease state or condition. Such preventing and suppressing need not be absolute to be useful.

The terms “treatment”, “treat” and “treating” as used herein refers a course of action (such as administering a compound or pharmaceutical composition) initiated after the onset of a clinical manifestation of a disease state or condition so as to eliminate or reduce such clinical manifestation of the disease state or condition. Such treating need not be absolute to be useful.

The term “in need of treatment” as used herein refers to a judgment made by a caregiver that a patient requires or will benefit from treatment. This judgment is made based on a variety of factors that are in the realm of a caregiver's expertise, but that includes the knowledge that the patient is ill, or will be ill, as the result of a condition that is treatable by a method, compound or pharmaceutical composition of the disclosure.

The term “in need of prevention” as used herein refers to a judgment made by a caregiver that a patient requires or will benefit from prevention. This judgment is made based on a variety of factors that are in the realm of a caregiver's expertise, but that includes the knowledge that the patient will be ill or may become ill, as the result of a condition that is preventable by a method, compound or pharmaceutical composition of the disclosure.

The term “individual”, “subject” or “patient” as used herein refers to any animal, including birds or mammals, such as mice, Norway rats, cotton rats, gerbils, cavies, hamsters, other rodents, rabbits, dogs, cats, swine, cattle, sheep, goat, horses, or primates, and humans. The term may specify male or female or both, or exclude male or female.

The term “therapeutically effective amount” as used herein refers to an amount of an agent, either alone or as a part of a pharmaceutical composition, that is capable of having any detectable, positive effect on any symptom, aspect, or characteristics of a disease state or condition. Such effect need not be absolute to be beneficial.

The term “including” as used herein is non-limiting in scope, such that additional elements are contemplated as being possible in addition to those listed; this term may be read in any instance as “including, but not limited to.”

The term “adhesion inhibiting effective amount” means an amount of an agent sufficient to decrease the rate of adhesion, eliminate the rate of adhesion, or reverse adhesion between a virus and a cell.

The terms “immunize”, “immunizing”, and “immunization” means to elicit an immune response in a subject against an organism, such as but not limited to, a virus or a bacterium, in order to provide some level of protection to a subject against later infection with the organism. As used herein, immunization may occur via exposure to the organism naturally or by human intervention. In certain embodiments, the organism is an influenza virus.

The term “actively immunize”, “actively immunizing”, and “active immunization” means to purposefully immunize a subject by exposing a subject to organism, such as but not limited to, a virus or a bacteria; such exposure may be carried out by exposing the subject to an intact organism, an attenuated organism, a portion of the organism, one or more antigens present on the organism or a combination of the foregoing. In certain embodiments, the organism is an influenza virus.

The term “passively immunize”, “passively immunizing”, and “passive immunization” means to provide antibodies against an organism, such as but not limited to, a virus or a bacteria, or a component of an organism to a subject without necessarily eliciting an immune response to the organism in the subject.

The term “vaccinate”, “vaccinating”, and “vaccination” means to actively immunize a subject by administering a vaccine to the subject.

All pronouns are intended to be given their broadest meaning. Unless stated otherwise, female pronouns encompass the male, male pronouns encompass the female, singular pronouns encompass the plural, and plural pronouns encompass the singular.

### II. Physico-Chemical Characteristics of IgY

Birds (such as laying-hens) are highly cost-effective as producers of antibodies compared with mammals traditionally used for such production. Avian antibodies have biochemical advantages over mammalian antibodies. Immunologic differences between mammals and birds result in increased sensitivity and decreased background in immunological assays; as well as high specificity and lack of complementary immune effects when administered to mammalian subjects. In contrast to mammalian antibodies, avian antibodies do not activate the human complement system nor will they react with rheumatoid factors, human anti-mouse IgG antibodies, staphylococcal proteins A or G, or bacterial and human Fc receptors. Thus avian antibodies offer many advantages over mammalian antibodies.

IgY behaves like a natural F(ab′)2 analogue but presents a larger Fc fragment. The Fc fragment of IgY consists of a Cv3 and Cv4 group, in contrast to the Fc fragment of mammalian IgG, which consists of a Cy2 and Cy3 group. The divergence in the structures of the Fc fragments confers significant advantages to the use of IgY over IgG.

A. Research and Diagnostic Antibodies

Antibodies available to research laboratories generally belong to one of the three main categories: mammalian monoclonal antibodies, mammalian polyclonal antibodies, and avian polyclonal antibodies. The species chosen for antibody production have usually been mammals, most frequently rabbits. One avian species from which antibodies are highly defined and easily accessible is the chicken. The major serum antibody in chicken is IgY, which is also actively transported to the egg in a manner similar to the placental transfer of IgG in mammals. The protection against pathogens that the immuno-incompetent newly hatched chick has is through transmission of antibodies from the mother via the egg. In the egg, IgY is found mainly in the egg yolk, whereas the concentration in egg white is very low. Avian IgA and IgM is found in the egg white in very low amounts.

B. Quantity and Efficiency

While the egg is still in the ovary, birds transfer their serum immune globulins into the yolk. IgM and IgA are transferred together with other proteins in the oviduct into the egg white. IgG (or IgY) in the egg follicle is passed in large amounts into the yolk. In chickens, the IgY concentration in the yolk is comparable to the concentration of IgY in the serum, sometimes approximately 6-13 mg/ml. A laying hen produces approximately five to six eggs per week with a yolk volume of approximately 15 ml per egg. Therefore, in one week a hen produces egg antibodies equivalent to about 75-90 ml of serum, or 150-180 ml of whole blood. This could be compared to an immunized rabbit, which yields approximately 20 ml whole blood per week. Only large mammals such as cows or horses can produce more antibodies than a laying hen. The blood collection procedure is time consuming and stressful for the animal. Furthermore, the cost of feeding and handling is considerably lower for a hen than for a rabbit, cow or horse.

C. Avoiding Interferences

A frequently used approach for the detection of antigens is to create a so-called sandwich assay (immobilized capture antibody, antigen, and labeled detection antibody). The antibodies in such assays are usually derived from mammals, and the samples to be tested are often mammalian serum or plasma. If anti-mammalian IgG antibodies are present in the samples they may simulate the behavior of the antigen by linking the detection antibody to the capture antibody, thus causing false positive reactions. Such false positive reactions occur in sandwich assays whether or not the assay utilizes mammalian polyclonal or monoclonal antibodies. The most well known of these anti-mammalian IgG antibodies is rheumatoid factor, which is an IgM antibody reacting with the Fc fragment of mammalian IgG. This occurs in patients rheumatoid arthritis, but rheumatoid factor can also be found in sera from patients with other diseases and in sera from healthy individuals. As the sensitivity of the assay increases, so will the interference by anti-IgG antibodies.

Another cause for the presence of anti-IgG antibodies is the in vivo use of heterophilic antibodies. Mammalian antibodies have been used in vivo for the treatment of patients increasingly in recent years. Mouse monoclonal antibodies are also used in vivo for diagnosis and treatment of patients. When such antibodies are given to the patient, the patient normally responds by producing human anti-mouse Ig antibodies (HAMA). The HAMA will react with mouse antibodies but also with structurally related proteins such as IgG of other mammals. The presence of HAMA can hinder the diagnostic and therapeutic effect of mammalian IgG. For example, HAMA might bind to mammalian antibodies and inactivate them, preventing the mammalian antibodies from therapeutically binding to target pathogens. The presence of HAMA might give false positive reactions with all types of sandwich assays based on mammalian antibodies. Chicken IgG have no immunological cross-reactivity with mammalian IgG and can thus be used to avoid interference due to rheumatoid factor HAMA. The cross-reactivity between different mammalian IgG may also cause problems in histochemical staining If a mammalian IgG is used as primary antibody in histochemistry, the secondary anti IgG antibody may also react with IgG in the mammalian tissue section, which will result in an increased background staining. This can be avoided if IgY is used as the primary antibody due to the lack of cross-reactivity between IgY and mammalian IgG.

D. Effects on Complement System

The complement system is a biochemical cascade which functions to clear pathogens from an organism. It is part of the innate immune system that is not adaptable and does not change over the course of an individual's lifetime. However, it can be recruited and brought into action by the adaptive immune system. The classical pathway of activation of the complement system is a group of blood proteins that mediate the specific antibody response. It is initiated when an antibody (IgG or IgM) binds to an antigen, and the bound antibody subsequently binds to the C1 complex. This triggers a regulatory cascade that ultimately activates the complex of proteins known as the “membrane attack complex” (MAC). The MAC binds to the surface of the target cell, creating a pore in the membrane, resulting in lysis.

A distinct advantage of avian IgY over mammalian antibodies is that IgY does not trigger the complement system, whereas IgG and IgM do.

Many immunological assays utilize mammalian capture antibodies bound to a solid phase. When a serum sample is added to the immobilized antibodies, the complement system in the samples is activated and the complement components are bound to the antibodies. This binding may block the antigen binding sites, and it has been shown that complement activation may interfere with antigen binding to the capture antibody and significantly reduce the number of positively reacting samples. The complement system is inactivated during storage. The standards used in immunological assay have usually been stored for some time and will thus have an inactive complement system, whereas the complement activity in the patient samples vary, but may be very high. This will cause an analytical error. IgY antibodies do not activate the human complement system and can thus be used to avoid this interference problem.

E. Fc-Receptor Interference

Receptors for the Fc domain of IgG provide an important link between specific humoral responses and the cellular branch of the immune system. The binding of IgG to a Fc-receptor may trigger many biological responses (including phagocytosis, endocytosis, antibody-dependent cellular cytotoxicity, inflammation, and enhancement of antigen presentation). Many of these responses are undesirable during a course of treatment or prevention.

Flow cytometry is widely used in clinical laboratories for the detection of cell surface proteins. The system utilizes labeled antibodies for the detection of specific cell markers. When the antibody reacts with the antigen, an immune complex is formed Immune complexes containing mammalian antibodies may interact with Fc or complement receptors on the cell, which can cause cell activation and changes in the expression of surface proteins. It has been shown that immune complexes containing mammalian antibodies, but not IgY, will cause erroneous results when measuring platelet activation.

In a similar vein, if IgG is introduced to a mammalian subject for the purpose of treating or preventing disease caused by a target pathogen, the therapeutic effect of the antibody may be reduced or eliminated by competitive binding to Fc-receptor. Furthermore, Fc-receptor mediating scavenging by macrophages can be a major obstacle to administering antibodies to subject; this is especially true in the pulmonary administration of aerosols (Dellamary et al., J. Controlled Release, 95(3):489-500 (2004)).

Further analytical problems exist when mammalian IgG is used to detect an antigen in a sample in which bacteria are also present. Some bacteria have IgG-binding membrane proteins. The best known of these proteins are Staphylococcus aureus protein A and streptococcal protein G. These proteins will bind the Fc portion of IgG from many mammalian species, but they will not bind IgY. If staphylococci or streptococci are present in the sample, these bacteria may bind the Fc portion of the antibody and cause a false positive reaction.

In a similar vein, if IgG is introduced to a mammalian subject for the purpose of treating or preventing disease caused by a target pathogen, the therapeutic effect of the antibody may be reduced or eliminated by competitive binding to bacterial Fc-receptor if bacteria are present.

F. Stability

Experience with egg yolk antibodies is that they are stable over time. IgY antibodies have been stored for over 10 years at 4° C. without any significant loss in antibody activity. IgY antibodies have also retained their activity after 6 months at room temperature or 1 month at 37° C. This stability is a major advantage in every application. For example, formulations of IgY for therapeutic applications can be stored in refrigerators indefinitely, and do not require dry ice or ultralow temperatures to ensure they retain their activity. IgY antibodies are also useful in immunoprecipitation assays in agar.

Bird eggs can be the source of vast number of antibodies to almost any immunogenic stimulant for preventative and therapeutic purpose.

The properties of avian eggs create potential immunological applications for IgY antibodies for diagnostic purposes, as reagents in clinical chemistry, as reagents in immunology, as adhesion-inhibiting agents, as agents of disease prophylaxis, and as agents of disease treatment. IgY antibodies have biochemical advantages over mammalian antibodies due to the phylogenetic differences between avian and mammalian species, resulting in increased sensitivity as well as decreased background in immunological assays.

### III. Antibody Compositions

The antibodies of the instant disclosure solve the problem of the short supply and high cost of antibodies targeted to influenza, and particularly to H1N1/09 and HPAIV-H5N1. They can be simply manufactured, as they are a natural product of the avian immune system, and do not require high-technology manufacturing infrastructure to produce. The antibodies are very stable and can be stored or transported under simple refrigeration in eggs or when isolated or purified. These factors combined solve the serious problem of limited access of high-quality antibodies and use of such antibodies in developing countries.

There is a high demand and short supply effective vaccine against influenza generally, and particularly to H1N1/09 and HPAIV-H5N1. This problem is addressed by providing avian antibodies targeted to these strains from a plentiful and very low cost source: bird eggs. The eggs may be from any species of bird that will produce strain-specific IgY antibodies upon immunization and subsequently lay eggs containing high concentrations of the antibodies. It is preferred that the bird is a domestic fowl, as domestic fowl are abundant and easy to raise. Domestic fowl include chicken, duck, swan, goose, turkey, peacock, guinea hen, ostrich, pigeon, quail, pheasant, and dove. In some embodiments of the composition, the bird is a chicken; chickens have the advantages of being the most numerous domestic fowl, and large numbers of chicken are vaccinated against one or more strains of influenza as a matter of routine.

The low-cost antibodies of the instant disclosure also provide for immunoassays for the detection and the diagnosis of influenza in a subject, such as influenza caused by H5N1 or H1N1.

In some embodiments of the composition, the composition comprises a constituent of a bird's egg, wherein the bird's egg comprises a therapeutically effective amount or an adhesion-inhibiting effective amount of IgY specific for an influenza strain, such H5N1 or H1N1. Crude egg yolk may be used as an antibody source. However, avian antibodies are usually purified or concentrated from the yolk prior to use. The constituent of the bird's egg may be concentrated or purified as necessary, as is understood by those skilled in the art. In some embodiments of the composition, the composition comprises the yolk of the egg, or any IgY antibody-containing fraction thereof. The yolk is preferable to the white of the egg, as the yolk typically contains much higher concentrations of IgY than does the white. However, the white may contain concentrations of IgY sufficient for some applications.

If the constituent is a yolk or yolk-fraction, it may be administered by any method known in the art. Such methods of administration include those described for pharmaceutical compositions below. Such methods additionally include the oral administration of the uncooked yolk or yolk-fraction of the egg, alone or in combination with the white of the egg. Oral administration of the raw yolk or fraction may be performed for example by eating, rinsing or gargling. The yolk or fraction may be administered in combination with other ingredients to make it more palatable or nutritious. Thus the yolk or fraction may be consumed as a food item; alternatively, the yolk or fraction may be consumed as part of a pharmaceutical composition. If it is consumed as a food item, it is preferably uncooked or very lightly cooked, as cooking can inactivate the antibody.

In some embodiments of the method, the antibody composition is a pharmaceutical comprising the contents of a bird egg, such as the contents of the bird egg comprising an adhesion-inhibiting effective amount of IgY-specific for influenza. The pharmaceutical may comprise additional components as discussed elsewhere in the disclosure. The pharmaceutical may be administered by any method known in the art. Some preferred methods of administration include as an intranasal or inhaled aerosol, as an oral/pharyngeal rinse, and as a gargle; such methods of administration provide efficient delivery to the respiratory tract, an area where influenza virus is likely to adhere to the cells of the subject or cause infection. Avian antibodies are superior to mammalian antibodies when administered in this manner, as discussed herein.

In some embodiments of the antibody composition, the IgY is concentrated, isolated, or purified from the constituent of the bird egg. This can be accomplished by a variety of methods. In some embodiments the antibodies may be purified by the water dilution method. The precipitate may then be removed by any conventional method, including centrifugation. The supernatant can then be stored frozen, for example at −20° C. IgY can then be isolated by precipitation with ammonium sulfate and subsequent dialysis. If desired, the titer of IgY antibodies can be determined by immunoassay, for example ELISA. The water dilution method is more completely described in the well-known literature, for example by Akita and Nakai (1993), which is incorporated by reference to teach this method. Other useful methods are described for example is U.S. Pat. No. 4,550,019, U.S. Pat. No. 4,748,018, and U.S. Patent Publication 2004/0161427 which are hereby incorporated by reference for such teachings. Commercial kits are available for example from the Promega Corporation (Madison, Wis.).

Some embodiments of the antibody composition are substantially isolated. In such embodiments a significant fraction of a non-antibody yolk component has been removed. The non-antibody yolk component may be for example the lipid component of the yolk, the carbohydrate component of the yolk, the yolk granules, the hydrophobic component of the yolk, the steroid component of the yolk, and the non-immunoglobin protein component of the yolk. The fraction of the component removed is at least 50%. In some embodiments the removed fraction is at least 60%, 75%, 80%, 90%, 95%, 99%, or 99.9%. Greater removed fractions have the advantage of producing a more pure antibody composition. Smaller removed fractions have the advantage of requiring less processing.

Some embodiments of the antibody composition are substantially concentrated. In such embodiments the concentration of IgY will be greater in the composition than in the egg yolk. Substantially concentrated antibody compositions comprise IgY that is at least twice as concentrated as in the yolk. Some embodiments of the substantially concentrated antibody composition are concentrated by at least a factor of 3, 4, 5, 6, 7, 8, 9, 10, 100, 1000, or 10,000. More concentrated antibody compositions have the advantage of providing the same mass of antibodies in lower volume. Less concentrated antibody compositions have the advantage of requiring less processing.

The antibody compositions of the present disclosure may be processed so as to largely remove all isotypes except IgG and IgY. In some embodiments of the pharmaceutical composition the immunoglobulin may be derived from numerous donors. Any number of donors may be used. In some embodiments, the antibodies are derived from one donor. In further embodiments, the antibodies are derived from 1-10 donors. In further embodiments, the antibodies are derived from 10-100 donors. In further embodiments, the antibodies are derived from 100-1000 donors. In still further embodiments, the antibodies are derived from over 1000 donors. Some embodiments of the antibody composition comprise polyclonal antibodies; some embodiments comprise monoclonal antibodies (in this context “monoclonal” does not refer to antibodies produced by a single B-cell cell line, but rather a set of monospecific antibodies). In embodiments of the antibody composition comprising monoclonal antibodies, the antibody fraction of the egg constituent may be further purified to select for a set of monospecific antibodies.

In some embodiments of the antibody composition, the composition is made by the method comprising obtaining an egg laid by a fowl previously immunized against influenza and separating the antibody fraction from a yolk of the egg. In some embodiments of the composition the fowl has been actively immunized, for example by vaccination. In other embodiments the fowl is immunized without human intervention (for example, immunized as a result of unintended infection). In some embodiments of the antibody composition, the antibody composition is made by the same method, further comprising acquiring the egg on an open market in a country in which vaccination of poultry against influenza is legally mandatory. The fowl is preferably a domesticated fowl. The domesticated fowl may be chicken, duck, swan, goose, turkey, peacock, guinea hen, ostrich, pigeon, quail, pheasant, dove, or other domesticated fowl. The domesticated fowl is preferably a chicken. The domesticated fowl is more preferably a domesticated chicken raised primarily for egg or meat production. The fowl may be immunized against any strain of influenza, any subtype of influenza, any type of influenza, or combinations thereof.

Use of eggs from chickens raised for egg or meat production, and which are vaccinated pursuant to this purpose, has the great advantage of using as the feedstock for the process eggs that are widely available commercially in great volumes and at very low price. Previously, animals used for the production of antibodies have been raised solely or mainly for that purpose, and maintained in small numbers at very high expense.

In some embodiments of the antibody composition, the antibody composition is made by a method comprising actively immunizing a hen against influenza, collecting eggs from the hen after an immunization period, and separating the antibody fraction from a yolk of the egg. Optionally, collecting eggs from the hen can occur continuously after the immunization period.

The immunization of the bird may occur by any means known in the art. For example, a vaccine may be administered to the bird that is known to effectively elicit an immune response in birds, or that is known to effectively elicit an immune response in mammals. Many such influenza vaccines are commercially available, and can be routinely developed by those of ordinary skill in the art without undue experimentation.

Further methods of producing IgY with a specific target are known to those skilled in the art. Such methods can be found for example in U.S. Pat. No. 4,550,019, U.S. Pat. No. 4,748,018, and U.S. Patent Publication 2004/0161427, and U.S. Pat. No. 6,537,500, which are incorporated by reference as necessary to enable those of ordinary skill in the art to make or practice the methods claimed herein without undue experimentation. The antibodies disclosed in this section are suitable for use in any of the methods and compositions described in this disclosure.

In one embodiment of the antibody composition, the method of making the composition comprises vaccinating the fowl using an antigen found in H1N1 A/PR8/34. It is now apparent that persons born before 1949 produce antibodies that are cross-reactive with H1N1/09. This is hypothesized to result from the exposure of this group to the strain H1N1 A/PR8/34, a strain first identified in 1934. As a result, fowl immunized with H1N1 A/PR8/34 can be used as a source of IgY that recognize H1N1/09.

In any composition or method disclosed herein, an antigen binding fragment of an IgY antibody such as an Fab or Fab2 fragment, may substitute for the IgY antibody. The antigen binding fragment may be any fragment that includes the antigen-binding region of the original IgY. In some embodiments of the compositions and methods, a modified version of an IgY antibody may substitute for the IgY antibody, so long as the antigen-binding region of the IgY antibody retains its ability to recognize the target strain of influenza.

In any composition or method described herein, an antigen binding fragment of an IgY antibody may substitute for the IgY antibody As used in the present disclosure, antigen binding fragments include Fv, Fab, Fab′ or other antigen binding portion of an antibody. Digestion of antibodies to produce fragments thereof, such as Fab fragments, can be accomplished using routine techniques known in the art. For instance, digestion can be performed using papain. Examples of papain digestion are described in WO 94/29348 published and U.S. Pat. No. 4,342,566, each of which are incorporated herein by reference for such teaching. Papain digestion of antibodies typically produces two identical antigen binding fragments, called Fab fragments, each with a single antigen binding site, and a residual Fc fragment. Pepsin treatment yields a fragment that has two antigen combining sites and is still capable of cross linking antigen.

The antigen binding fragments, whether attached to other sequences, also include insertions, deletions, substitutions, or other selected modifications of particular regions or specific amino acids residues, provided the activity of the antibody or antigen binding fragment is not significantly altered or impaired compared to the non-modified antibody or antigen binding fragment. These modifications can provide for some additional property, such as to remove/add amino acids capable of disulfide bonding, to increase its bio-longevity, to alter its secretory characteristics, etc. In any case, the antibody or antigen binding fragment must possess a bioactive property, such as specific binding to its cognate antigen. Functional or active regions of the antibody or antigen binding fragment may be identified by mutagenesis of a specific region of the protein, followed by expression and testing of the expressed polypeptide.

The IgY of the antibody composition may recognize any strain, type, or subtype of influenza. For example, the IgY may recognize influenza Type A. Some embodiments of the IgY recognize influenza Type A comprising a hemagglutinin subtype selected from the group consisting of: H1, H2, H3, H5, H7, H9, and H10. Some embodiments of the IgY recognize a subtype selected from the group consisting of H1N1, H1N2, H2N2, H3N2, H5N1, H7N2, H7N3, H7N7, H9N2, and H10N7. Particular embodiments of the IgY recognize at least one of H1N1, H1N1/09, H5N1, and HPAIV-H5N1.

### IV. Pharmaceutical Compositions

Useful compositions of the present disclosure may comprise one or more antibodies useful in the treatment and prevention methods of the present disclosure, such as, but not limited to, antibodies specific for influenza. A pharmaceutical composition for the prevention or treatment of influenza is provided, comprising a therapeutically effective amount of any antibody composition disclosed herein.

The compositions disclosed may comprise one or more of such antibodies or antibody compositions disclosed above, in combination with a pharmaceutically acceptable carrier. Examples of such carriers and methods of formulation may be found in Remington: The Science and Practice of Pharmacy (20^{th} Ed., Lippincott, Williams & Wilkins, Daniel Limmer, editor). To form a pharmaceutically acceptable composition suitable for administration, such compositions will contain a therapeutically effective amount of an antibody. The therapeutically effective amount may be an adhesion inhibiting effective amount.

The pharmaceutical compositions of the disclosure may be used in the treatment and prevention methods of the present disclosure. Such compositions are administered to a subject in amounts sufficient to deliver a therapeutically effective amount of the antibody so as to be effective in the treatment and prevention methods disclosed herein. The therapeutically effective amount may vary according to a variety of factors such as, but not limited to, the subject's condition, weight, sex and age. Other factors include the mode and site of administration. The pharmaceutical compositions may be provided to the subject in any method known in the art. Exemplary routes of administration include, but are not limited to, subcutaneous, intravenous, topical, epicutaneous, oral, intraosseous, intramuscular, intranasal and pulmonary. Intranasal and pulmonary administration may be achieved using aerosols (solid or liquid). The aerosols may be of any known formulation, including spray-dried lipid microparticles, formulated with or without an acceptable surfactant (for example, see the immunoglobulin aerosols of Dellamary et al., J. Controlled Release, 95(3):489-500 (2004); and Hill et al., Infection & Immunity, 74(5): 3068-3070 (2006)). The aerosol may be delivered by any means known to those skilled in the art, including via a nebulizer, an atomizer, a sprayer, or by gargling a liquid.

The compositions of the present disclosure may be administered only one time to the subject or more than one time to the subject. Furthermore, when the compositions are administered to the subject more than once, a variety of regimens may be used, such as, but not limited to, one per day, once per week, once per month or once per year. The compositions may also be administered to the subject more than one time per day. The therapeutically effective amount of the antibody and appropriate dosing regimens may be identified by routine testing in order to obtain optimal activity, while minimizing any potential side effects. In addition, co-administration or sequential administration of other agents may be desirable.

The compositions of the present disclosure may be administered systemically, such as by intravenous administration, or locally such as by subcutaneous injection or by application of a paste or cream.

The compositions of the present disclosure may further comprise agents which improve the solubility, half-life, absorption, etc. of the antibody. Furthermore, the compositions of the present disclosure may further comprise agents that attenuate undesirable side effects and/or or decrease the toxicity of the antibodies(s). Examples of such agents are described in a variety of texts, such a, but not limited to, Remington: The Science and Practice of Pharmacy (20^{th} Ed., Lippincott, Williams & Wilkins, Daniel Limmer, editor).

The compositions of the present disclosure can be administered in a wide variety of dosage forms for administration. For example, the compositions can be administered in forms, such as, but not limited to, nasal aerosols, aqueous rinses, an oral/pharyngeal rinse, a gargle, tablets, capsules, sachets, lozenges, troches, pills, powders, granules, elixirs, tinctures, solutions, suspensions, elixirs, syrups, ointments, creams, pastes, emulsions, or solutions for intravenous administration or injection. Other dosage forms include administration transdermally, via patch mechanism or ointment. Further dosage forms include formulations suitable for delivery by nebulizers or metered dose inhalers. Any of the foregoing may be modified to provide for timed release and/or sustained release formulations.

In the present disclosure, the pharmaceutical compositions may further comprise a pharmaceutically acceptable carrier. Such carriers include, but are not limited to, vehicles, adjuvants, surfactants, suspending agents, emulsifying agents, inert fillers, diluents, excipients, wetting agents, binders, lubricants, buffering agents, disintegrating agents and carriers, as well as accessory agents, such as, but not limited to, coloring agents and flavoring agents (collectively referred to herein as a carrier). Typically, the pharmaceutically acceptable carrier is chemically inert to the active antibodies and has no detrimental side effects or toxicity under the conditions of use. The pharmaceutically acceptable carriers can include polymers and polymer matrices. The nature of the pharmaceutically acceptable carrier may differ depending on the particular dosage form employed and other characteristics of the composition.

For instance, for oral administration in solid form, such as but not limited to, tablets, capsules, sachets, lozenges, troches, pills, powders, or granules, the antibody may be combined with an oral, non-toxic pharmaceutically acceptable inert carrier, such as, but not limited to, inert fillers, suitable binders, lubricants, disintegrating agents and accessory agents. Suitable binders include, without limitation, starch, gelatin, natural sugars such as glucose or beta-lactose, corn sweeteners, natural and synthetic gums such as acacia, tragacanth or sodium alginate, carboxymethylcellulose, polyethylene glycol, waxes and the like. Lubricants used in these dosage forms include, without limitation, sodium oleate, sodium stearate, magnesium stearate, sodium benzoate, sodium acetate, and the like. Disintegrators include, without limitation, starch, methyl cellulose, agar, bentonite, xanthum gum and the like. Tablet forms can include one or more of the following: lactose, sucrose, mannitol, corn starch, potato starch, alginic acid, microcrystalline cellulose, acacia, gelatin, guar gum, colloidal silicon dioxide, croscarmellose sodium, talc, magnesium stearate, calcium stearate, zinc stearate, stearic acid as well as the other carriers described herein. Lozenge forms can comprise the active ingredient in a flavor, usually sucrose and acacia or tragacanth, as well as pastilles comprising the active ingredient in an inert base, such as gelatin and glycerin, or sucrose and acadia, emulsions, and gels containing, in addition to the active ingredient, such carriers as are known in the art.

For oral liquid forms, such as but not limited to, tinctures, solutions, suspensions, elixirs, syrups, antibodies of the present disclosure can be dissolved in diluents, such as water, saline, or alcohols. Furthermore, the oral liquid forms may comprise suitably flavored suspending or dispersing agents such as the synthetic and natural gums, for example, tragacanth, acacia, methylcellulose and the like. Suitable coloring agents or other accessory agents can also be incorporated into the mixture. Other dispersing agents that may be employed include glycerin and the like.

Formulations suitable for parenteral administration include aqueous and non-aqueous, isotonic sterile injection solutions, which can contain anti-oxidants, buffers, bacteriostats, and solutes that render the formulation isotonic with the blood of the patient, and aqueous and non-aqueous sterile suspensions that can include suspending agents, solubilizers, thickening agents, stabilizers, and preservatives. The antibody may be administered in a physiologically acceptable diluent, such as a sterile liquid or mixture of liquids, including water, saline, aqueous dextrose and related sugar solutions, an alcohol, such as ethanol, isopropanol, or hexadecyl alcohol, glycols, such as propylene glycol or polyethylene glycol such as poly(ethyleneglycol) 400, glycerol ketals, such as 2,2-dimethyl-1,3-dioxolane-4-methanol, ethers, an oil, a fatty acid, a fatty acid ester or glyceride, or an acetylated fatty acid glyceride with or without the addition of a pharmaceutically acceptable surfactant, such as, but not limited to, a soap, an oil or a detergent, suspending agent, such as, but not limited to, pectin, carbomers, methylcellulose, hydroxypropylmethylcellulose, or carboxymethylcellulose, or emulsifying agents and other pharmaceutical adjuvants.

Oils, which can be used in parenteral formulations, include petroleum, animal, vegetable, or synthetic oils. Specific examples of oils include peanut, soybean, sesame, cottonseed, corn, olive, petrolatum, and mineral. Suitable fatty acids for use in parenteral formulations include polyethylene sorbitan fatty acid esters, such as sorbitan monooleate and the high molecular weight adducts of ethylene oxide with a hydrophobic base, formed by the condensation of propylene oxide with propylene glycol, oleic acid, stearic acid, and isostearic acid. Ethyl oleate and isopropyl myristate are examples of suitable fatty acid esters. Suitable soaps for use in parenteral formulations include fatty alkali metal, ammonium, and triethanolamine salts, and suitable detergents include (a) cationic detergents such as, for example, dimethyldialkylammonium halides, and alkylpyridinium halides, (b) anionic detergents such as, for example, alkyl, aryl, and olefin sulfonates, alkyl, olefin, ether, and monoglyceride sulfates, and sulfosuccinates, (c) nonionic detergents such as, for example, fatty amine oxides, fatty acid alkanolamides, and polyoxyethylene polypropylene copolymers, (d) amphoteric detergents such as, for example, alkylbeta-aminopropionates, and 2-alkylimidazoline quaternary ammonium salts, and (e) mixtures thereof.

Suitable preservatives and buffers can be used in such formulations. In order to minimize or eliminate irritation at the site of injection, such compositions may contain one or more nonionic surfactants having a hydrophile-lipophile balance (HLB) of from about 12 to about 17.

Topical dosage forms, such as, but not limited to, ointments, creams, pastes, emulsions, containing the antibodies of the present disclosure, can be admixed with a variety of carrier materials well known in the art, such as, e.g., alcohols, aloe vera gel, allantoin, glycerine, vitamin A and E oils, mineral oil, PPG2 myristyl propionate, and the like, to form alcoholic solutions, topical cleansers, cleansing creams, skin gels, skin lotions, and shampoos in cream or gel formulations. Inclusion of a skin exfoliant or dermal abrasive preparation may also be used. Such topical preparations may be applied to a patch, bandage or dressing for transdermal delivery or may be applied to a bandage or dressing for delivery directly to the site of a wound or cutaneous injury.

The antibody of the present disclosure can also be administered in the form of liposome delivery systems, such as small unilamellar vesicles, large unilamellar vesicles and multilamellar vesicles. Liposomes can be formed from a variety of phospholipids, such as cholesterol, stearylamine or phosphatidylcholines. Such liposomes may also contain additional monoclonal antibodies to direct delivery of the liposome to a particular cell type or group of cell types.

The antibody of the present disclosure may also be coupled with soluble polymers as targetable drug carriers. Such polymers can include, but are not limited to, polyvinyl-pyrrolidone, pyran copolymer, polyhydroxypropylmethacryl-amidephenol, polyhydroxyethylaspartamidephenol, or polyethyl-eneoxidepolylysine substituted with palmitoyl residues. Furthermore, the antibodies of the present invention may be coupled to a class of biodegradable polymers useful in achieving controlled release of a drug, for example, polylactic acid, polyepsilon caprolactone, polyhydroxy butyric acid, polyorthoesters, polyacetals, polydihydro-pyrans, polycyanoacrylates and cross-linked or amphipathic block copolymers of hydrogels.

In the formulation of passive immunizing agents in pharmaceuticals, the formulation may be optimized to obtain a certain desired titer of immunoglobulin in the subject and to minimize adverse reactions in the subject.

Some embodiments of the pharmaceutical composition are simple vaccines that are specific to a single pathogen; in other embodiments the pharmaceutical composition is a complex vaccine that is specific to many pathogens. In the latter case, additional antibodies may be present from any source. The range of influenza strains recognized by the antibodies of the composition may be as narrow as a single strain, or it may encompass many strains of influenza. In some embodiments the pharmaceutical composition is specific for at least one of H5N1, HPAIV-H5N1, H1N1, H1N1/09, and a combination thereof. In further embodiments, the pharmaceutical composition is specific to the A subtype of influenza. In further embodiments, the pharmaceutical composition is specific for one or more of the following subtypes: H1, H2, H3, H5, H7, H9, and H10. In further embodiments, the pharmaceutical composition is specific for one or more of the following subtypes: H1N1, H1N2, H2N2, H3N2, H5N1, H7N2, H7N3, H7N7, H9N2, and H10N7. In further embodiments, the pharmaceutical composition is specific for one or more of the following variants: bird flu, human flu, swine flu, horse flu, and dog flu.

The pharmaceutical compositions of the present disclosure may be modified to prevent adverse reactions in the subject. Such potential adverse reactions include serum sickness, host recognition, anaphylaxis, localized inflammation and other forms of allergic reaction. Adverse reactions are more common in heterologous antibody treatment than in homologous antibody treatment, although the advantages of avian antibodies in this respect have been explained. In some embodiments of the pharmaceutical composition, the antibody is modified to alter the Fc region of the molecule. In further embodiments, the antibody is treated to prevent binding between the Fc region of the antibody and the Fc receptor of a cell.

The pharmaceutical preparations of the present disclosure can be stored in any pharmaceutically acceptable form, including an aqueous solution, a frozen aqueous solution, a lyophilized powder, or any of the other forms described herein.

### V. Viral Adhesion Inhibitors

It has been discovered that IgY antibodies from bird eggs are a cheap and plentiful source of viral adhesion inhibitors. Such antibodies bind to the surface of an antigen-bearing virus (such as an influenza virus), thus preventing the initial stages of contact between the virus and a potential host cell. As explained elsewhere in this disclosure, preventing the initial stages of adhesion between a virus and a host cell has numerous applications, including treatment of viral disease and prevention of viral disease.

Provided is a viral adhesion inhibitor comprising an adhesion inhibiting effective amount of any antibody composition described herein.

### VI. Methods of Inhibiting Viral Adhesion to Cells, and Kits Therefore

Methods are provided for inhibiting or preventing viral adhesion to a cell. The first step in the infection of a cell by a virus is contact and adhesion between virus and cell. Although this step is critical to the establishment of infection, methods of preventing infection at this early stage are few. More typically viral infection is countered using techniques such as vaccination, which causes the body to produce antibodies that trigger a cell-mediated immune response, or antiviral chemotherapy. If vaccination is not feasible, most often viral disease is merely treated symptomatically. The methods described here offer an effective means to prevent this early step in the infection process without requiring administration well in advance of the subject's exposure to the pathogen, as is required by vaccination.

Antibodies can function to prevent adhesion between virus and cell by binding to the virus and interfering with the ability of the virus to bind its target membrane receptor. Avian antibodies (such as IgY) have distinct advantages over mammalian antibodies in this application, particularly when the subject is a mammal. As stated above, the advantages of avian antibodies include that avian antibodies as compared to mammalian antibodies are more specific, more stable, and cause fewer unwanted forms of immune response. Avian antibodies can also be easily and cheaply obtained from eggs.

A method of inhibiting viral adhesions to a cell is provided, comprising contacting the virus with an adhesion-inhibiting amount of any of the antibody compositions disclosed herein. Some embodiments of the method, the method comprises administering to a subject an adhesion-inhibiting effective amount of any viral adhesion inhibitor disclosed herein.

In some embodiments of the method, the cell is part of an ex vivo cell culture or system. In other embodiments of the method, the cell is part of an intact and living subject. The subject may be any organism, for example an animal. The subject may be an animal that is susceptible to influenza, including any of the strains, types, and subtypes of influenza disclosed herein. The subject is more preferably a bird or a mammal. If the subject is a mammal, it can be any species, including human. In some embodiments of the method, the subject is a domesticated bird or mammal. The domesticated bird or mammal can be of any domesticated species or breed thereof, but is preferably a species that is susceptible to influenza (even as a carrier or vector). The domesticated bird or mammal may be a swine, cattle, goat, sheep, rabbit, domesticated fowl, dog, cat, cavy, other livestock animal, or other domesticated animal. Other animals known to be susceptible to influenza include wild fowl, seals, ferrets, camels, house cats, minks, and horses.

If the cell is part of an intact living organism, any method of administration of the inhibitor or pharmaceutical can be used that is known in the art or described herein. For example, in some embodiments of the method the antibody composition contacts the influenza virus in the respiratory tract of the subject. Accordingly, in some embodiments administration is by inhalation, rinsing, gargling, or swallowing the viral adhesion inhibitor or pharmaceutical composition. In some embodiments of the method contact between antibody and virus occurs in an ex vivo environment, such as a surface or medium suspected of having virus present. In such embodiments the antibody composition may be supplied as a spray, a rinse, a powder, or any other form known in the art for carrying antiviral agents.

Kits for inhibiting the adhesion of a virus to a cell are provided, comprising a packaged volume of an adhesion-inhibiting effective amount of any of the antibody compositions disclosed herein or any of the viral adhesion inhibitors disclosed herein. Instructions for the use of the kit may also be provided.

### VII. Methods of Treatment and Prevention of Influenza, and Kits Therefore

The introduction to a subject of antibodies specific for a pathogen of concern is known as “passive immunization.” Despite the name, passive immunization can be used both prophylactically and curatively. Vaccination (a form of active immunization) involves the introduction of a pathogen-associated antigen to the subject, and the subsequent development by the subject's immune system of antibodies and immune cells specific for the antigen. Vaccination typically involves a protracted period of time between the administration of the vaccine to the subject and the acquisition by the subject of immunity. Passive immunization introduces antibodies that bind to the pathogen, without necessarily inducing the subject's own immune system to produce antibodies and immune cells targeted to the pathogen. Because the subject's immune system requires no time to develop a response to an antigen, passive immunization can be used effectively to treat a subject that is already sick or has already been exposed to the pathogen. If used preventatively, passive immunization is effective immediately.

As described herein, avian antibodies are of particular utility in passive immunization. Compared to mammalian antibodies they are highly specific, do not cross react, and do not cause unwanted immune reactions.

A method of treatment or prevention of influenza in a subject in need thereof comprising administering to the subject a therapeutically effective amount of any of the pharmaceutical compositions disclosed herein. It is understood that the IgY component of the pharmaceutical will recognize at least one strain, type, or subtype of influenza of which the subject is of need of treatment or prevention. The method may further comprise identifying the subject in need of treatment or prevention of influenza. In some embodiments of the method, administration occurs prior to the onset of a symptom of influenza. In some embodiments of the method, administration occurs concurrently with or after the onset of a symptom of influenza.

The pharmaceutical compositions may additionally contain any appropriate additional constituent or be administered by any appropriate means, as described herein or as understood by those skilled in the art. In one embodiment, the administration to the subject may be oral, pulmonary, intranasal, or nasopharyngeal. In a specific embodiment, the administration to the subject is by means of an aerosol. The aerosol may be generated by any appropriate means, including using a nebulizer, inhaler (such as a dry powder inhaler), atomizer, or gargle.

A kit for the treatment or prevention of influenza is also provided, comprising a packaged dosage of any of the antibody compositions and/or pharmaceutical compositions disclosed herein. Instructions for the use of the kit may also be provided.

### VIII. Methods of Detection and Diagnosis

The antibody compositions of the instant disclosure are useful as reagents in immunoassays for the detection and diagnosis of influenza. Methods of detecting an influenza virus are provided, comprising performing an immunoassay on a sample, the immunoassay comprising contacting a sample with a constituent of a bird egg, the constituent comprising an IgY that recognizes a strain of influenza. Some embodiments of the immunoassay comprise any of the reporter antibodies disclosed herein; in such embodiments the IgY component of the antibody composition may be conjugated to a detectable reagent. However, other non-conjugated IgY may be present. Some embodiments of the method comprise contacting the sample with any of the antibody compositions disclosed herein. The method may be performed in vivo or ex vivo.

Such assay methods include, but are not limited to, radioimmunoassays, immunohistochemistry assays, in situ hybridization assays, competitive-binding assays, Western Blot analyses, ELISA assays and proteomic approaches, two-dimensional gel electrophoresis (2D electrophoresis) and non-gel based approaches such as mass spectrometry or protein interaction profiling. Assays also include, but are not limited to, competitive and non-competitive assay systems using techniques such as radioimmunoassays, enzyme immunoassays (EIA), enzyme linked immunosorbent assay (ELISA), sandwich immunoassays, precipitin reactions, gel diffusion reactions, immunodiffusion assays, agglutination assays, complement-fixation assays, immunoradiometric assays, fluorescent immunoassays, protein A immunoassays, and immunoelectrophoresis assays. IgY may particularly be used as secondary antibodies in radial immunodiffusion, agar gel double immunodiffusion, immunoelectrophoresis, rocket immunoelectrophoresis two-dimensional (Crossed) immunoelectrophoresis and ELISAS. Additionally IgY may be labeled with fluorochromes and enzymes for further immunofluorescent, immunohistochemical and immunochemical assays. For examples of immunoassay methods, see U.S. Pat. No. 4,845,026 and U.S. Pat. No. 5,006,459.

In an ELISA assay, an antibody is prepared, if not readily available from a commercial source, specific to an antigen, such as, for example, an influenza antigen. In addition, a reporter antibody generally is prepared. The reporter antibody comprises an IgY recognizing influenza, and is attached to a detectable reagent such as a radioactive, fluorescent or enzymatic reagent, for example horseradish peroxidase enzyme or alkaline phosphatase. In one embodiment of the ELISA, to carry out the ELISA, antibody specific to antigen is incubated on a solid support that binds the antibody. Any free protein binding sites on the dish are then covered by incubating with a non-specific protein. Next, the sample to be analyzed is incubated with the solid support, during which time the antigen binds to the specific antibody. Unbound sample is washed out with buffer. A reporter antibody specifically directed to the antigen and linked to a detectable reagent is introduced resulting in binding of the reporter antibody to any antibody bound to the antigen. Unattached reporter antibody is then washed out. Reagents for detecting the presence of the reporter antibody are then added. The detectable reagent is then determined in order to determine the amount of antigen present. In an alternate embodiment, the antigen is incubated with the solid support, followed by incubation with one or more antibodies, wherein at least one of the antibodies comprises a detectable reagent. Quantitative results may be obtained by reference to a standard curve.

A reporter antibody is provided comprising an IgY conjugated to a detectable reagent, wherein the IgY recognizes a strain of influenza. In some embodiments, the detectable reagent is selected from the group consisting of: a radionuclide, a fluorochrome, a microsphere, a ferromagnetic particle, a secondary antigen, and an enzyme. In some embodiment of the reporter antibody, the IgY is an IgY component of any of the antibody compositions disclosed herein.

### IX. Animal Study Demonstrating Efficacy

Egg yolk immunoglobulins (IgY) have been used mainly for treatment of infectious diseases of gastrointestinal tract, however effectiveness of IgY against influenza virus infection has not been explored. It is reported here that virus-specific IgY isolated from eggs of immunized hens protected mice infected with A/H1N1 (A/PR8/34) model virus. When administered intranasally before or after lethal infection, IgY provided prevention or significantly reduced virus replication resulting in complete recovery from the disease, respectively. The effectiveness of IgY was further examined against highly pathogenic avian influenza A virus (HPAIV) strain A/H5N1 that caused extensive damages to poultry producers and remains a serious threat to global health. We found that marketed chicken eggs in Vietnam, where mass poultry vaccination against A/H5N1 is mandatory, contain high levels of virus-specific IgY that provide prevention against and therapy of infections with HPAIV A/H5N1 and related A/H5N2 strains. Thus, virus-specific IgY offer potentially inexpensive yet highly safe and effective alternative for prevention and treatment against current A/H1N1 and potential HPAIV A/H5N1 pandemics.

### A. IGY-MEDIATED PROTECTION AGAINST INFECTION WITH LETHAL DOSE OF THE MOUSE-ADAPTED A/PR/8/34 VIRUS

The protective effect of IgY isolated from eggs laid by hens immunized in the laboratory with heat-inactivated human influenza A H1N1 virus, A/PR/8/34, was examined. PR/8 virus is a common laboratory mouse-adapted influenza strain that can be handled safely under BSL2 conditions. Substantial levels of hemagglutination inhibition (HAI) and virus neutralization (VN) Abs were found in the sera and yolks derived from immunized hens (Table 1).

TABLE 1

| | Sera | | IgY | |
|---|---|---|---|---|
| Immunization | HAI (log₂) | VN | HAI (log₂) | VN |
| Heat inactivated A/PR8 | 6* | 9.3* | 8* | 8.3* |
| Inactivated A/Goose/GD/96 | 5.3 ± 1.5 | ND | 7 and 6** | 7.3** |
| consumable eggs from Vietnam - | | | | |
| batch 1 | | | | |
| Inactivated A/Goose/GD/96 | 5.5 ± 1.0 | ND | 7 | ND |
| consumable eggs from Vietnam - | | | | |
| batch 2 | | | | |
| Unimmunized - consumable eggs | <2 | ND | <2 | <1:10 |
| from Korea | | | | |

ND: Not done
*A/PR/8/34 (PR8)
**A/Vietnam/1203/2004 (VN/1203)

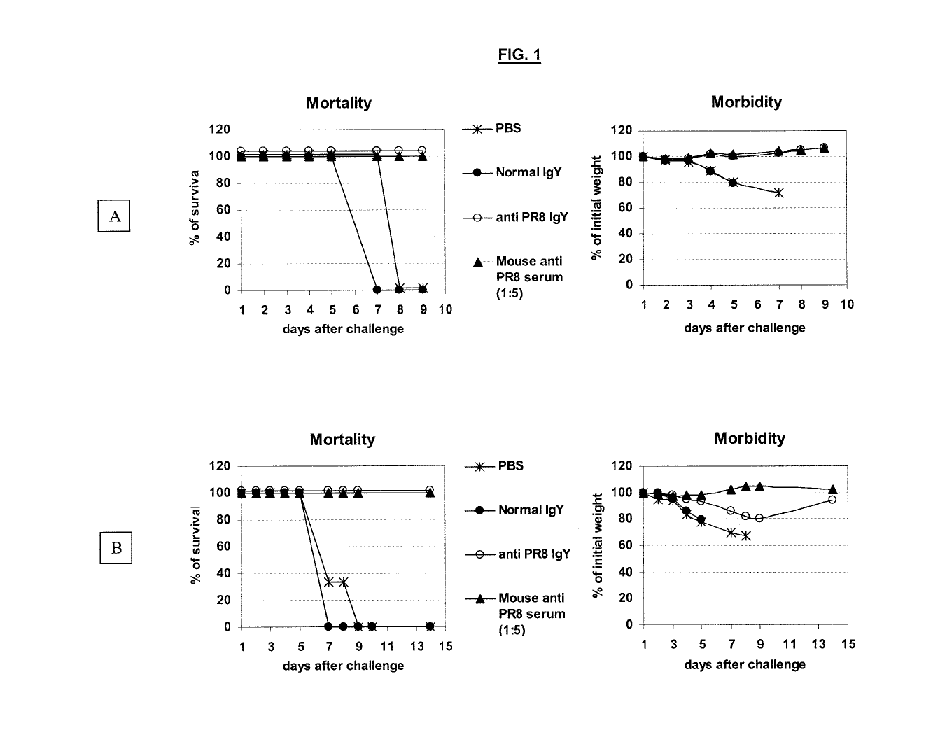

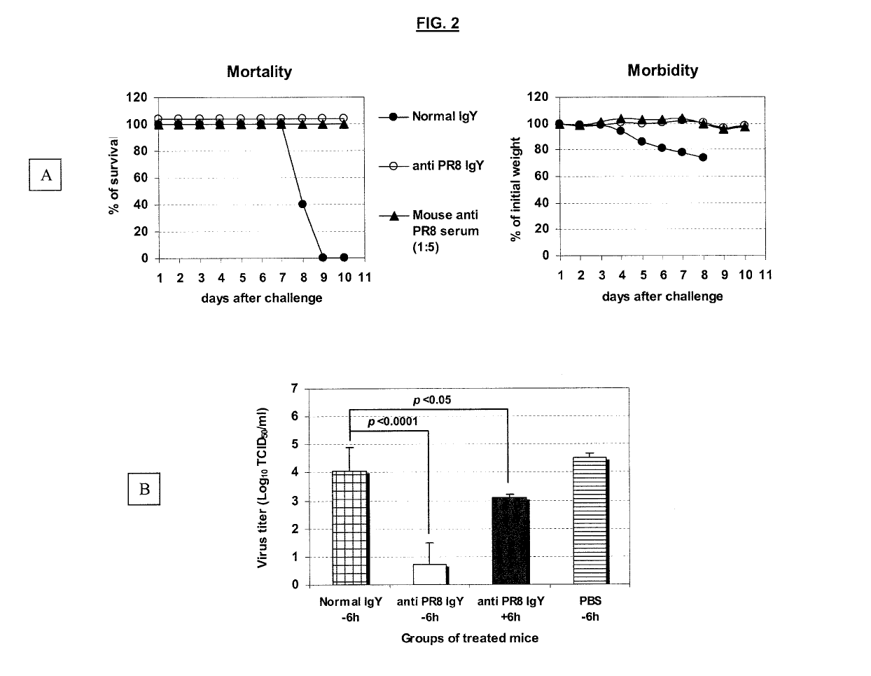

When naïve mice were administered intranasally with such anti-PR/8 IgY at 6-8 h before (FIG. 1(A)) or after infection with lethal dose of PR/8 virus (FIG. 1(B)), they were protected from the infection or lethal disease, respectively. Importantly, a single treatment at 6 h before the lethal infection prevented weight loss, a measure of morbidity, which was comparable with that seen in the control group receiving murine immune serum specific for A/PR8 virus (anti-PR/8 serum) (FIG. 2(A)). The virus titers in the lungs of A/PR8 specific IgY-treated mice at day 3 after infection were significantly lower than those seen in untreated mice or mice receiving normal IgY (FIG. 2(B)). Oral or intraperitoneal treatments with such IgY did not provide protection, although IgY was detectable by conventional ELISA in the sera of IgY-treated mice after oral or intraperitoneal delivery (data not shown). These results show that virus-specific IgY prevents influenza virus infection and cure the disease when such IgY is applied intranasally. The protection is correlated with virus neutralization (VN) activity of the virus-specific IgY and virus clearance in the lungs of infected mice.

### B. HAI AND VN ACTIVITIES OF IGY ISOLATED FROM CONSUMABLE EGGS AVAILABLE IN MARKETS

It was determined whether yolks from commercially available eggs in Vietnam contain H5N1-specific IgY. First, HAI titers were determined in the sera and yolks of the eggs obtained from a farm in Vietnam that was participating in a national mass vaccination program. IgY preparation was restored in PBS to the original volume of yolk. HAI titers determined in yolks were comparable to those seen in sera of immunized hens (Table 1). The HAI titers were determined of IgY isolated from eggs purchased in randomly selected supermarkets in Hanoi, Vietnam that offer safe foods with recorded origin. Consistently, 90% of eggs purchased in supermarkets contain H5-specific IgY at the levels comparable with those observed in sera of hens selected randomly from the farm that underwent supervised H5N1 vaccination. Similar VN titers were found in IgY preparations derived from eggs purchased in Vietnam. In contrast, IgY separated from eggs laid by unimmunized hens or purchased in Korean markets where poultry are not vaccinated against avian influenza H5N1 has no detectable H5-specific HAI or VN activity.

### C. PROTECTION AGAINST INFECTION WITH LETHAL DOSE OF HIGHLY PATHOGENIC H5 INFLUENZA VIRUSES

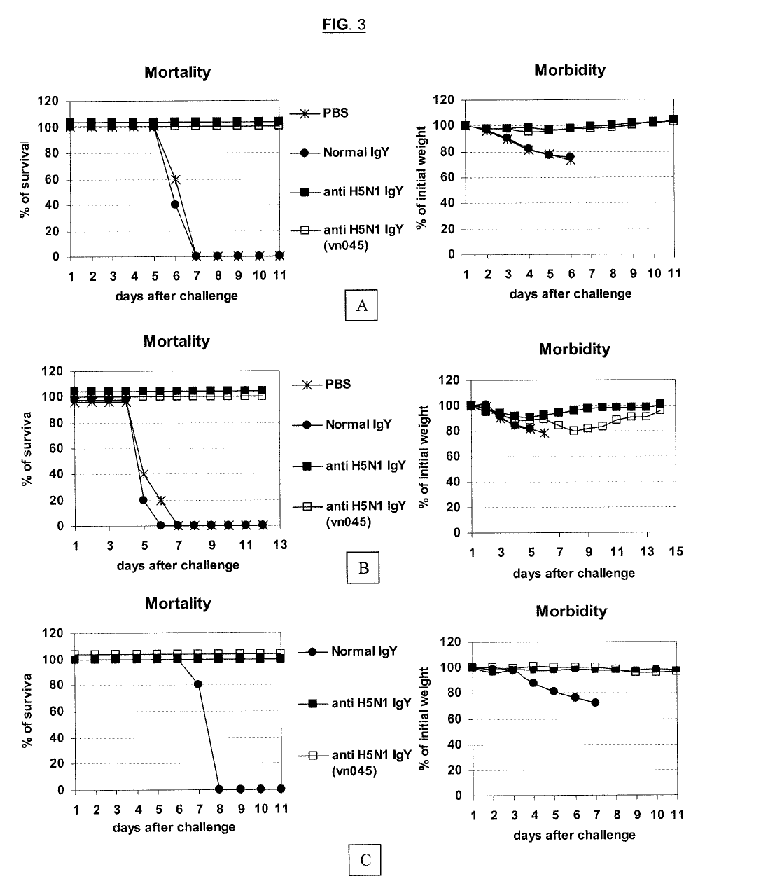

A mouse-adapted low pathogenic avian influenza A virus (LPAIV) strain A/Aquatic bird/Korea/W81/2005 (H5N2) was used that shares 94.4% nucleotide sequence homology with HA (H5) but different NA (N2) from the one used for mass immunization in Vietnam (reassortant avian H5N1 influenza virus A/Goose/GD/96-derived, strain Re-1) for challenge experiments. As shown in FIG. 3, complete protection against infection with avian H5N2 was achieved by intranasal administration with H5N1-specific IgY before or after the lethal infection (FIGS. 3(A) and 3(B)). A single treatment with H5N1-specific IgY before inoculation was sufficient to protect animals completely from disease (FIG. 3(C)).

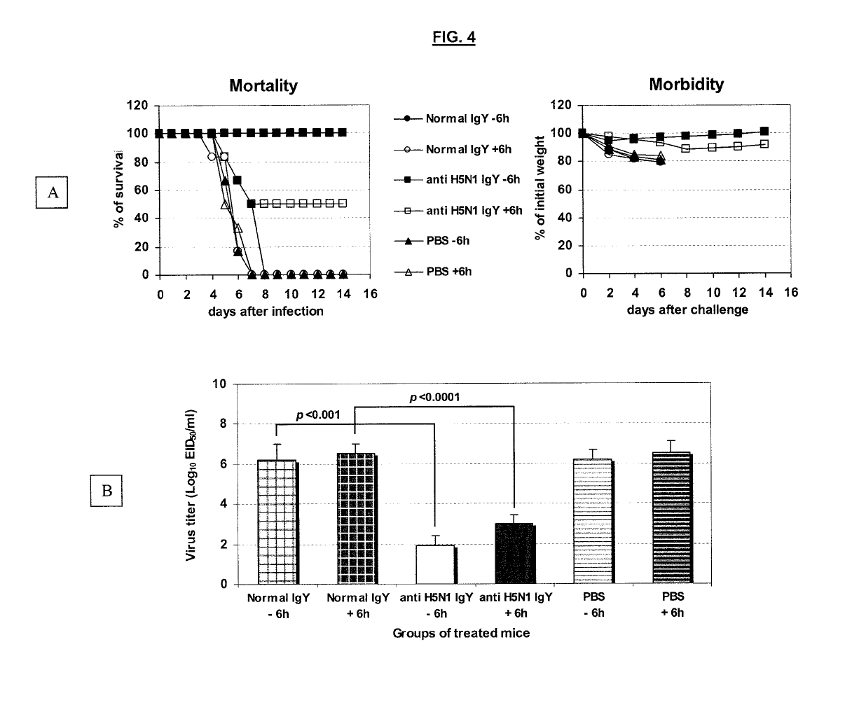

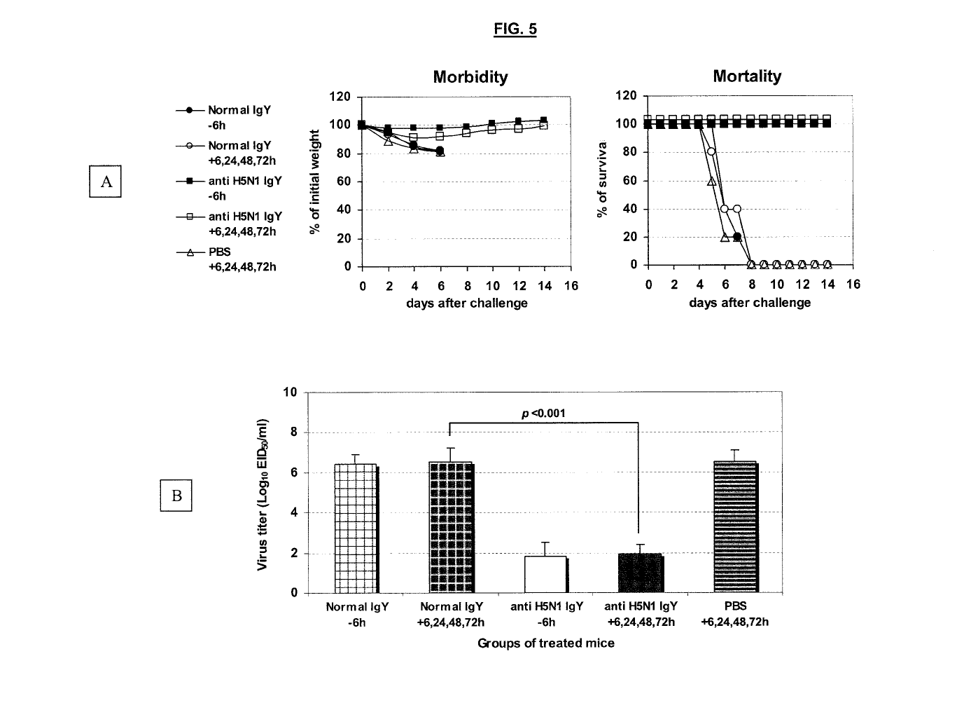

Based on these results, it was further examined whether protection against infection with HPAIV-H5N1 strain, A/Vietnam/1203/2004, isolated from a fatal case could be achieved. Animals treated intranasally with H5N1-specific IgY before infection displayed mild weight loss and recovered completely by the end of the first week after inoculation (FIG. 4(A)). Of note, animals treated with H5N1-specific IgY after H5N1 inoculation exhibited minimal weight loss during the first week after inoculation (FIG. 4(A)) and virus titers in the lungs were substantial reduced at day 3 after infection (FIG. 4(B)), but 50% of treated mice succumbed to infection during the second week after inoculation (FIG. 4(A)). It is possible that not all the HPAIV-H5N1 viruses were neutralized upon the single treatment with IgY and escaping viruses can spread systemically to organs outside of the lungs. These viruses may reappear in lung tissue later when specific IgY is absent. Indeed, VN/1203 virus injected intravenously or into the brain can spread to the lungs (13). To circumvent the virus escape, we administered multiple post-infection treatments with H5N1-specific IgY. As a result, all infected mice recovered completely by the second week post-infection (FIG. 5(A)) and virus titers in the lungs were substantially reduced to the level that seen in protected mice that received single pre-infection treatment (FIG. 5(B)). The results indicate that H5N1-specific IgY isolated from eggs purchased in markets have preventive and therapeutic effects against infection with HPAIV H5N1 and the related strain H5N2. The findings suggest that while a single treatment with IgY prior to lethal infection was sufficient to protect the animals from the infection, multiple treatment is required for complete therapeutic effect after infection with HPAIV such as VN/1203 strain. The protection was correlated with HAI and virus-neutralizing activities of the IgY and reduced virus titers in the lungs after treatments suggesting that the VN is the major mechanism by which the IgY mediates the protection.

### D. ANTI-IGY ABS GENERATED IN MICE TREATED WITH IGY

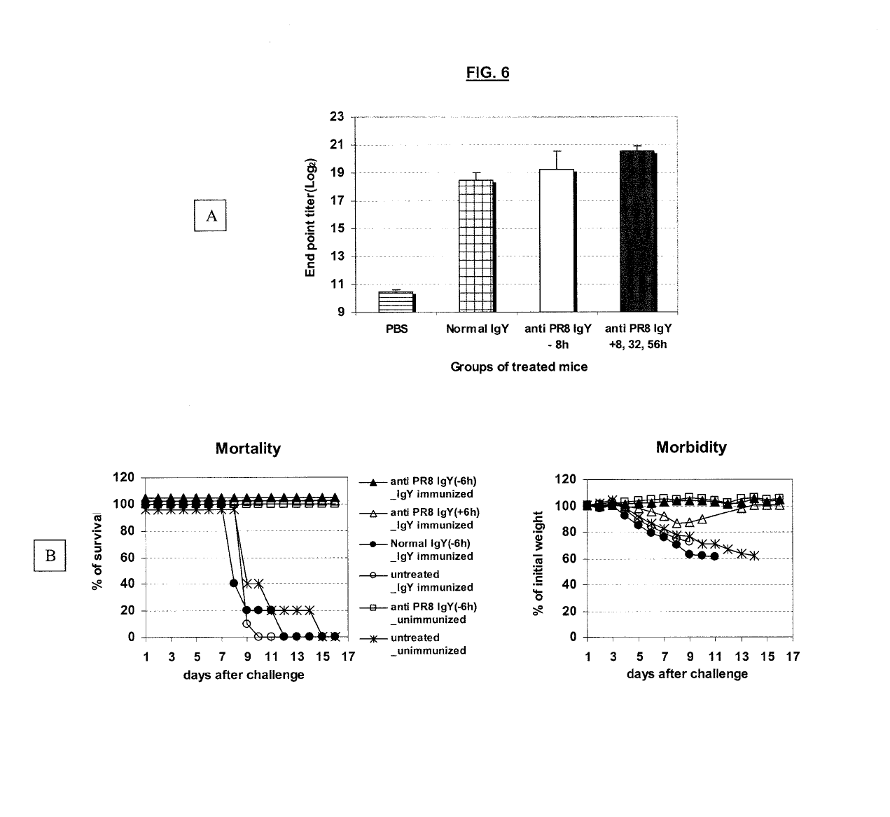

Although raw eggs are consumed widely in many countries and uncooked egg components are used in preparation of many foods including a popular dessert named Tiramisu, report on presence of anti-IgY in humans is limited in two studies. One demonstrated the presence of anti-IgY in sera obtained from normal individuals (14) and other showed the absence of anti-IgY Abs in humans upon oral ingestion of IgY or consumption of raw egg components (15). In mice, intravenous injection of IgY elicits typical anti-IgY antibody response (16). It is, however, not clear if administration of IgY in the respiratory tract induces anti-IgY response. If this is the case, it raises a question whether pre-existing anti-IgY Abs have impact on the protective effect of virus specific IgY-mediated treatment. We examined sera obtained from IgY treated mice for presence of anti-IgY antibodies. Indeed, high levels of anti-IgY were observed in animals that received IgY from only one intranasal administration (FIG. 6(A)). There was no significant difference in the levels of anti-IgY in mice receiving multiple or single administration of IgY.

### E. PRE-EXISTING ANTI-IGY DOES NOT PREVENT PROTECTION MEDIATED BY VN IGY

It was then determined whether pre-existing anti-IgY antibodies prevent virus specific IgY-mediated protection. If this were to occur, it may affect treatment of individuals who have already anti-IgY antibodies or the need for multiple IgY-treatments during individuals' lifetime. Mice with normal IgY or immune IgY specific for particular subtypes were immunized. Three weeks later, serum Abs specific for IgY were determined by ELISA. One hundred percent of immunized mice generated anti-IgY Abs at the level comparable to that of IgY-treated mice (FIG. 6(A)). Such IgY-immunized mice were then treated with virus-specific IgY before or after infection with lethal dose of influenza virus. The results are almost identical to those obtained from treated unimmunized mice (FIG. 6(B)) indicating that pre-existing anti-IgY Abs do not interfere the protection mediated by virus-specific IgY. The finding suggests that the IgY treatment could be applied to persons who have developed anti-IgY during the individuals' life and such treatment strategy could be repeated if multiple treatment is required and necessary later on to protect infections with other pathogens.

### F. ANTI-IGY ABS DO NOT BLOCK HAI OR VN ACTIVITIES OF THE VIRUS-SPECIFIC IGY

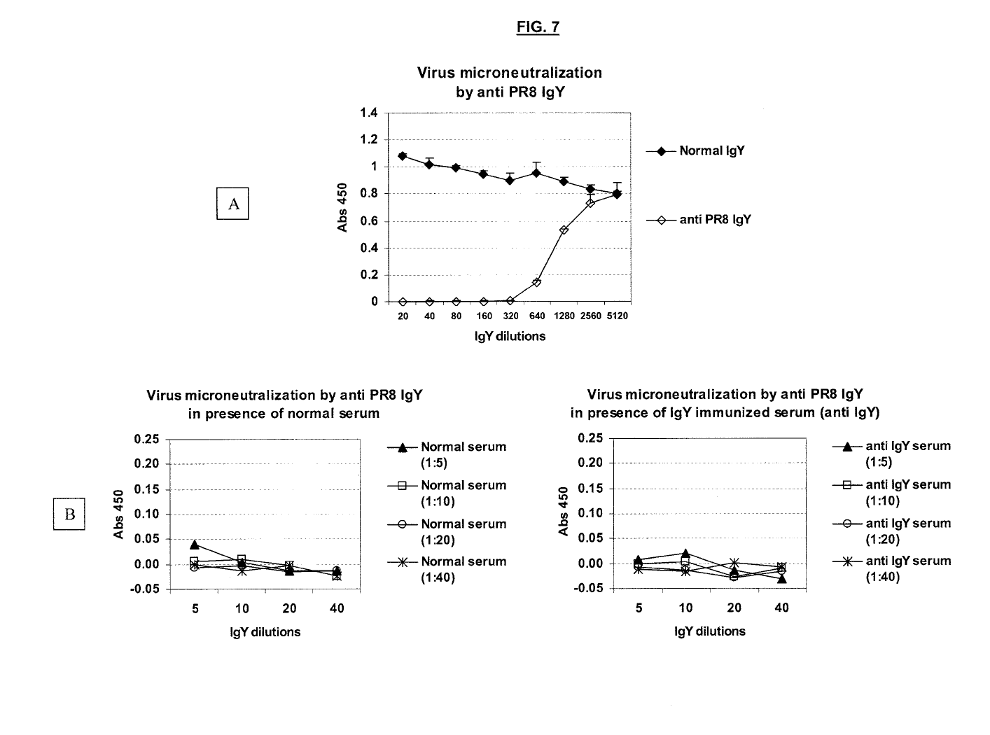

It was speculated that, if IgY epitopes that bind anti-IgY Abs are not located in the virus-binding sites of the IgY, then anti-IgY Abs would not prevent the binding and/or neutralizing activities of virus-specific IgY. To investigate this question, murine anti-IgY serum was incubated with virus-specific IgY before adding to the HAI and VN assays. Indeed, incubation with anti-IgY serum did not interfere with HAI activity of the virus-specific IgY (not shown) indicating that anti-IgY Abs do not block virus binding by virus-specific IgY. Similarly, the incubation with anti-IgY does not interfere with VN activity of the specific IgY (FIG. 7).

### G. CONCLUSIONS

The approach using specific IgY for prevention and therapy of HPAIV H5N1 infection offers a practical alternative to immunotherapy using convalescent plasma (17) and an additional therapeutic option to antiviral drugs since widespread drug resistance has been recently reported among influenza virus strains. Current FDA-approved anti-influenza virus drugs consist of the adamantane compounds (amantidine/rimantidine) and the neuraminidase inhibitors oseltamivir and zanamivir (18, 19). Widespread adamantine resistance was documented among seasonal H1N1 and H3N2 strains and a majority of clade 1 and some clade 2 H5N1 isolates from Southeast Asia (20-23). Oseltamivir-resistant H5N1 and H1N1 isolates have also been reported (24-26).

These studies demonstrate that influenza virus-specific IgY can be used in passive immunization against influenza Importantly, the consumable eggs readily available in the markets of countries that impose mandatory H5N1 vaccination offer an enormous source of valuable, affordable and safe biological material for prevention and protection against potential H5N1 pandemic influenza.

### H. METHODS

1. Animals

Female wild-type (WT) BALB/cAnNCrl (H-2d) mice were purchased at 6 to 8 weeks of age from Charles River Co. (Wilmington, Mass.) or The Jackson Laboratory (Bar Harbor, Me.). All mice were maintained in specific pathogen-free barrier facilities. All experiments and animal procedures conformed to protocols approved by the Institutional Animal Care and Use Committees of Seoul National University, Yonsei University, Konkuk University, Seoul, Korea, and Centers for Diseases Control and Prevention (US CDC), Atlanta, Ga., USA. Hy-Line Leghorn hens purchased from Kyunggi Poultry Farm were housed in animal facility at Konkuk University. All the hens were kept in rooms lightened for 16 h per day with constant temperature of 25° C.

2. Cell Lines

Madin-Darby canine kidney (MDCK) cells (ATCC, Manassas, Va.), were maintained in standard complete Dulbecco's modified Eagle's medium (D-MEM) (Gibco, Grand Island, N.Y.) containing 5% fetal bovine serum (FBS) and antibiotics.

3. Viruses

Influenza virus strains A/PR/8/34 (H1N1) (A/PR8), A/Philippines/2/82/X-79 (H3N2) (A/Philippines), were prepared as previously reported (31). Mouse-adapted viruses A/PR8 and A/Philippines prepared as lung homogenates of intranasally infected mice were used for challenge. The H5N1 human influenza isolate A/Vietnam/1203/2004 (VN/1203) was obtained from the World Health Organization (WHO) influenza collaborating laboratory at the Centers for Disease Control (CDC), Atlanta, Ga. Inactivated reassortant avian H5N1 influenza virus (A/Goose/GD/96-derived, strain Re-1) (Harbin, China) was used for mass vaccination of poultry in Vietnam and A/ck/Scotland/59 (H5N1) was used for determination of haemagglutination inhibition (HAI) titers of sera and IgY from hens raised in Vietnam. The A/Aquatic bird/Korea/W81/2005 (H5N2), isolated from wild bird in Korea in 2006, kindly provided by Dr. Young-Ki Choi, Chungbuk University, Korea, was adapted by multiple passages (15 times) in BALB/c mice. After final passage, single plaque was isolated by three consecutive plaque purifications on MDCK cells, amplified in embryonated chicken eggs, and the LD₅₀ of the H5N2 virus was determined in mice for challenge experiment. Avian H5N1 viruses were propagated in the allantoic cavity of 10-day-old embryonated hen's eggs at 37° C. for 24 h to 30 h. The H5N1 human influenza isolate was incubated for an additional 10 h to 18 h. Allantoic fluid was pooled from multiple eggs, clarified by centrifugation, and frozen at −70° C. until use. All experiments with HPAI (VN1203) virus were conducted under biosafety level 3 containment, including enhancements (BSL3+) required by the U.S. Department of Agriculture and the Select Agent (2).

4. Eggs

Eggs laid by hen raised in the poultry unit of Konkuk University, Seoul, Korea and purchased from randomly selected supermarkets in Hanoi, Vietnam and Seoul, Korea and farms in Vietnam were used in experiments.

5. Hen Immunization

Twenty-five-week-old domestic Leghorn hens were immunized intramuscularly with heat-inactivated A/PR8/34 (H1N1) mixed with Freund's adjuvant (FA) (Sigma, Mo., USA). 5 μg of antigen was suspended in 250 μl of phosphate-buffered saline (PBS) and emulsified with an equal volume of complete FA. Incomplete FA was used for boosting immunizations. The hens were immunized three times with 2 weeks between the immunizations. The sera were collected eight weeks after the initial immunization and eggs laid after last immunization were collected continuously. In some cases, immunized hens were boosted within 3-4 months interval to keep in hyperimmunized condition for a longer time period.

6. Preparation of IgY

A rapid and simple water dilution method for extraction of IgY from egg yolk was adapted from the work by Akita and Nakai (33). Briefly, the yolk from ten eggs (total volume, 120 ml) was separated from the white by egg separators and washed with deionized water. Each yolk sac was disrupted by inserting a needle and the yolk was allowed to drip through a nylon mesh into a measuring cylinder. The egg yolk was diluted 10 times with cold 3 mM HCl to give the suspension a final pH of 5 (adjusted with 10% acetic acid). The suspension was incubated for at least 6 h at 4° C. before the supernatant containing the IgY was collected by centrifugation (10 000×g for 15 min at 4° C.). Solid ammonium sulfate was added to reach 60% saturation (390 g/l) and the mixture was stirred in the cold for 15 min Precipitate was collected by centrifugation and washed once with 60% saturated ammonium sulfate (SAS). The protein precipitate was dissolved in PBS and dialyzed three times against at least 10 volumes of PBS. Dialyzed IgY was adjusted to the original egg yolk volume (ten yolks equal 120 ml) pasteurized at 600 C for 30 minutes and stored at 40 C. The purity of the IgY preparations was determined by sodium dodecyl sulfate-polyacrylamid gel electrophoresis (SDS-PAGE).

7. Infection and Treatment of Mice

Fifty percent lethal dose (LD₅₀) titers were determined by inoculating groups of eight mice i.n. with serial 10-fold dilutions of virus as previously described (34). For infection, ketamine-anesthetized mice were inoculated intranasally with a lethal dose with 250 pfu (5×LD₅₀) of A/PR/8/34 (H1N1) virus, 1,000 pfu (5×LD₅₀) of A/Philippines (H3N2), 10×LD₅₀ of VN/1203 (H5N1) or 5×LD₅₀ A/Aquatic bird/Korea/W81/2005 (H5N2) resuspended in 50 μl PBS per animal. Ketamine-anesthetized mice were treated intranasally with 50 μA of IgY before or after infection.

8. Virus Titration

The 50% egg infectious dose (EID₅₀) was determined by serial titration of virus stock in eggs, and EID₅₀/ml values were calculated according to the method of Reed and Muench (35). Human virus stocks were grown in MDCK cells as described previously (36), with viral titers determined by standard plaque assay. The 50% tissue culture infectious dose (TCID₅₀) of virus was determined by titration in MDCK cells (37). Briefly, freshly trypsinized MDCK cells were adjusted to 2.0×10⁵/ml in Dulbecco's modified Eagle's medium containing 1% bovine serum albumin and antibiotics (V diluent) and 100 μl was added to each well in 96-well NUNCLON™ Surface plates (NUNC, Inc., Roskilde, Denmark) The plates were incubated for 3 h at 37° C. and 5% CO₂ and washed once with serum free medium. ½-log dilutions of virus in 100 μl of V diluent containing 2.5 μg/ml of trypsin were added to each well and incubated for 18 h at 37° C. and 5% CO₂. Plates were washed with PBS and fixed in cold 80% acetone in PBS for 10 min. The plates were subsequently washed three times with PBS containing 0.05% Tween 20. The anti-NP antibody of murine origin (US CDC, Atlanta, Ga.) diluted 1/4,000 in PBS containing 1% bovine serum albumin and 0.1% Tween 20 (E diluent) was added to each well and the plates were incubated at room temperature for at least 1 h. The plates were washed four times with PBS containing 0.05% Tween 20, and 100 μl of horseradish peroxidase-labeled goat anti-mouse immunoglobulin G (IgG) (Southern Biotech, Birmingham, Ala.) diluted 1/5,000 in E diluent was added to each well. The plates were incubated for at least 1 h at room temperature and then washed six times with wash buffer; 100 μg of freshly prepared TMB (3,3′,5,5′-tetramethylbenzidine) substrate (BD Biosciences, Franklin Lakes, N.J.) was added to each well, and the plates were incubated at room temperature for approximately 5 min. The reaction was stopped with 50 μl of 1 M sulfuric acid. The absorbance was measured at 450 nm (A₄₅₀) with SPECTRAmax photometer (Molecular Devices, Palo Alto, Calif.). Wells having an absorbance reading greater than 3 standard deviations above the mean absorbance of wells containing only MDCK cells were scored positive for virus growth. The TCID₅₀ of each stock virus was calculated by the method of Reed and Muench(35).

9. ELISA

The standard ELISA was performed for detection of anti-IgY in the sera of IgY-immunized mice. 96-well MaxiSorp™ Nunc-Immuno plates (Nalgene Nunc International, Naperville, Ill.) were coated overnight with purified IgY (Gallus Immunotech, Ontario, Canada) at a concentration of 0.5 μg/ml. Dilutions of serum were incubated 2 h on coated and blocked ELISA plates. Bound immunoglobulins were detected with horseradish peroxidase-conjugated donkey anti chicken IgY (Gallus Immunotech, Ontario, Canada). At the end of the incubation (2 h at 37° C.), TMB substrate was added and the reaction was stopped with an equal volume of 1 N sulfuric acid. The color developed was measured in a SPECTRAmax photometer at 450 nm. The reproducibility of the assay was ascertained by applying on each plate a control hyperimmune mouse serum. Assay results were expressed as end-point titration values.

10. Statistics

The data are expressed as the mean±one standard error of the mean (SEM) and compared using a two-tailed student's t-test or an unpaired Mann Whitney U test available in Microsoft Excel software (Redmond, Wash.).

### I. REFERENCES

1. H. J. Ehrlich et al., N. Engl. J. Med. 358, 2573 (2008).
2. M. A. Keller, E. R. Stiehm, Clin. Microbiol. Rev. 13, 602 (2000).
3. C. P. Simmons et al., PLoS Med. 4, e178 (2007).
4. A. P. Lim et al., Virol. J. 5, 130 (2008).
5. B. J. Hanson et al., Respir. Res. 7, 126 (2006).
6. L. Sun et al., PLoS One 4, e5476 (2009).
7. G. W. Warr, K. E. Magor, D. A. Higgins, Immunol. Today 16, 392 (1995).
8. D. Carlander, J. Stalberg, A. Larsson, Ups. J. Med. Sci. 104, 179 (1999).
9. A. Larsson, D. Carlander, Ups. J. Med. Sci. 108, 129 (2003).
10. E. Nilsson, A. Larsson, H. V. Olesen, P. E. Wejaker, H. Kollberg, Pediatr. Pulmonol. 43, 892 (2008).
11. M. Peyre, G. Fusheng, S. Desvaux, F. Roger, Epidemiol. Infect. 137, 1 (2009).
12. Anonymous, paper presented at the An OIE/FAO/IZSVe scientific conference, Verona, Italy, Mar. 20-22 2007.
13. K. J. Szretter et al., J. Virol. 83, 5825 (2009).
14. M. W. Russell, J. Mestecky, B. A. Julian, J. H. Galla, J. Clin. Immunol. 6, 74 (1986).
15. A. Larsson, A. Karlsson-Parra, J. Sjoquist, Clin. Chem. 37, 411 (1991).
16. W. E. Walsh, Jr. et al., Immunology 101, 467 (2000).
17. L. K. Kong, B. P. Zhou, Hong Kong Med. J. 12, 489 (2006).
18. R. L. Tominack, F. G. Hayden, Infect. Dis. Clin. North Am. 1, 459 (1987).
19. L. V. Gubareva, L. Kaiser, F. G. Hayden, Lancet 355, 827 (2000).
20. Anonymous, Emerg. Infect. Dis. 11, 1515 (2005).
21. R. A. Bright et al., Lancet 366, 1175 (2005).
22. C. L. Cheung et al., J. Infect. Dis. 193, 1626 (2006).
23. V. M. Deyde et al., J. Infect. Dis. 196, 249 (2007).
24. M. D. de Jong et al., N. Engl. J. Med. 353, 2667 (2005).
25. Q. M. Le et al., Nature 437, 1108 (2005).
26. T. G. Sheu et al., Antimicrob. Agents Chemother. 52, 3284 (2008).
27. A. Larsson, R. M. Balow, T. L. Lindahl, P. O. Forsberg, Poult. Sci. 72, 1807 (1993).
28. H. Kollberg et al., Pediatr. Pulmonol. 35, 433 (2003).
29. E. Nilsson, J. Hanrieder, J. Bergquist, A. Larsson, J. Agric. Food Chem. 56, 11638 (2008).
30. J. Kovacs-Nolan, M. Phillips, Y. Mine, J. Agric. Food Chem. 53, 8421 (2005).
31. H. H. Nguyen, F. W. van Ginkel, H. L. Vu, J. R. McGhee, J. Mestecky, J. Infect. Dis. 183, 368 (2001).
32. J. Y. Richmond, R. W. McKinney, Laboratory biosafety level criteria. J. Y. Richmond, R. W. McKinney, Eds., Biosafety in microbiological and biomedical laboratories. (Center for Disease Control and Prevention, Atlanta, Ga., ed. 5th, 2007).
33. E. M. Akita, S, Nakai, J. Food. Sci. 57, 629 (1992).
34. T. R. Maines et al., J. Virol. 79, 11788 (2005).
35. L. J. Reed, H. A. Muench, Am. J. Hyg. 27, 493 (1938).
36. T. M. Tumpey et al., Science 310, 77 (2005).
37. T. Rowe et al., J. Clin. Microbiol. 37, 937 (1999).

### X. Conclusions

The foregoing description illustrates and describes the processes, machines, manufactures, compositions of matter, and other teachings of the present disclosure. Additionally, the disclosure shows and describes only certain embodiments of the processes, machines, manufactures, compositions of matter, and other teachings disclosed, but, as mentioned above, it is to be understood that the teachings of the present disclosure are capable of use in various other combinations, modifications, and environments and is capable of changes or modifications within the scope of the teachings as expressed herein, commensurate with the skill and/or knowledge of a person having ordinary skill in the relevant art. The embodiments described hereinabove are further intended to explain certain best modes known of practicing the processes, machines, manufactures, compositions of matter, and other teachings of the present disclosure and to enable others skilled in the art to utilize the teachings of the present disclosure in such, or other, embodiments and with the various modifications required by the particular applications or uses. Accordingly, the processes, machines, manufactures, compositions of matter, and other teachings of the present disclosure are not intended to limit the exact embodiments and examples disclosed herein.

1. An antibody preparation comprising a constituent of a bird egg, the constituent comprising an adhesion inhibiting effective amount of an IgY that recognizes a strain of influenza.

2-59. (canceled)
